(19) United States
(12) Patent Application Publication
(10) Pub. No.: US 2009/0022827 A1
(43) Pub. Date: Jan. 22, 2009

(54) AGENT AND METHOD FOR ELIMINATING MALIGNANCE OF CANCER CELLS WITHOUT HARMFUL EFFECT TO NORMAL CELLS

(76) Inventors: Ming Li, Hong Kong (CN); Lei Cheng, Hong Kong (CN)

Correspondence Address: WANG & HO, 66 HILLTOP ROAD, MILLINGTON, NJ, 07946, US

(21) Appl. No.: 12/176,441

(22) Filed: Jul. 21, 2008

Related U.S. Application Data
(60) Provisional application No. 60/950,609, filed on Jul. 19, 2007.

(51) Int. Cl. A61K 36/73; A61P 35/04

(52) U.S. Cl. 424/765

(57) ABSTRACT

A pharmaceutical composition including an extract from Geum japonicum and a method of treating cancer using the extract. In vitro and in vivo experimental data demonstrated that the extract can induce selectively apoptosis of cancer cells and kill the cancer cells without affect normal cells.

### CROSS REFERENCE TO RELATED APPLICATIONS

This application claims priority to U.S. Provisional Patent Application No. 60/950,609, filed Jul. 19, 2007, the contents of which are hereby incorporated by reference.

### FIELD OF THE INVENTION

This invention relates to a pharmaceutical composition and a method of treating cancer. Particularly, it relates to a pharmaceutical composition comprising an extract from Geum japonicum and method for killing malignant cells without affecting healthy cells.

### BACKGROUND OF THE INVENTION

Next to heart diseases, in 2007, more than 1.4 million new cancer cases were diagnosed and nearly 560,000 cancer deaths occur in the United States alone. Currently, the most commonly used cytotoxic cancer therapeutic agents eliminated tumors by inducing apoptosis and inhibiting proliferation of rapid growing cells including both malignant and normal cells, therefore the severe side-effects of using anti-tumor chemotherapy. Ideally, a next generation of anti-cancer drug could specifically kill malignant cells or turn off the ability of malignant cells to multiply and spread without affecting the normal healthy cells.

### SUMMARY OF THE INVENTION

It was indicated that the number and sources of anticancer and anti-infective agents have reached more than 60% of the approved drugs developed in these diseases that can trace their lineage back to a natural product structure [1-3]. Plants have a long history of use in the treatment of cancer and other diseases [1-4]. We have found that an ethanol extract (GJ-B) obtained from Geum japonicum showed potent cytotoxic effects to several different tumor cells without any detectable toxicity to normal cells or tissues at the same dosage both in vitro and in several tumor animal models. The ethanol extract was further fractionated by a bio-assay guided fractionation strategy and a sub-fraction (GJ-B-3) containing approximately 15 compounds was isolated, which showed potent anti-cancer effects in a dose-dependent manner both in vitro (cancer cell culture systems) and in vivo (cancer animal models). Further studies demonstrated that both ethanol extract and the sub-fraction from ethanol extract can induce tumor cells significant apoptosis both in vitro and in vivo but showing almost no-apoptotic inductive effects on normal cells or tissues.

The various features of novelty which characterize the invention are pointed out with particularity in the claims annexed to and forming a part of this disclosure. For a better understanding of the invention, its operating advantages, and specific objects attained by its use, reference should be made to the drawings and the following description in which there are illustrated and described preferred embodiments of the invention.

### BRIEF DESCRIPTION OF DRAWINGS

FIG. 1 outlines the process of isolating GJ-B-3 from Geum Japonicum.

FIG. 2 shows the effect of GJ-B-3 in inducing apoptosis of cultured HepG2 cells.

FIG. 3 shows the inhibitory effect of GJ-B-3 on cancer.

FIG. 4 shows the effect of GJ-B-3 in inducing apoptosis of inoculated cancer cells in vivo.

FIG. 5 shows the appearance of the tumor cells under scan electron microscope.

### DETAILED DESCRIPTION OF PARTICULAR EMBODIMENTS

All protocols used in the present invention conformed to the Guide for the Care and Use of Laboratory Animals published by the U.S. National Institutes of Health, and were approved by the Animal Experimental Ethical Committee of The Chinese University of Hong Kong.

I. Isolation of GJ-B-3 from Geum japonicum

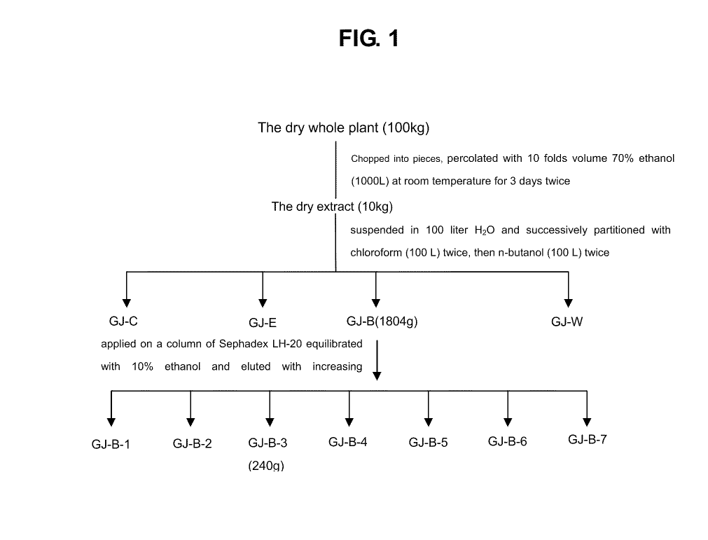

For the experiments disclosed in the following, the active fraction of Geum japonicum (GJ-B-3), which showed the anti-cancer effect, was isolated from the plant of Geum japonicum. Referring to FIG. 1, the plant, which was collected from Zhangjiajie, Hunan Province of China in the summer season (July), was dried (100 kg) and percolated with 10 folds volume 70% ethanol (1000 L) at room temperature for 3 days twice. The extract was combined and spray-dried to yield a solid residue (10 kg). The solid residue was suspended in 100 liter H₂O and successively partitioned with chloroform (100 L) twice, then n-butanol (100 L) twice to produce the corresponding fractions. The n-butanol (GJ-B) soluble fraction was filtered and spray dried to yield a powder fraction. The n-BuOH soluble fraction was applied on a column of Sephadex LH-20 equilibrated with 10% ethanol and eluted with increasing concentration of ethanol in water, resolving 7 fractions. Fraction 3 (GJ-B-3), eluted with approximately 50% ethanol, showed the potent activity in anti-cancer.

### II. GJ-B-3 Induced Apoptosis of Cancer Cells In Vitro

Six cancer cell lines including PC3 (prostate cancer), MCF7 (breast cancer), HepG2 and SK-HEP-1 (liver cancer), MKN28 (gastric cancer), and H292 (lung cancer) were tested with GJ-B-3 in vitro and rat bone marrow-derived mesenchymal stem cells and C2C12 (Mouse myoblast cell line) were used as the normal control. It was shown by live cell imaging that GJ-B-3 started to kill the cultured cancer cells at about 12-18 hours (“h” hereinafter) post treatment at the concentration of 20 ug GJ-B-3/ml, and 60-80% cancer cells could be killed within 48 h. After 48 h treatment, the remaining viable cancer cells seemed having lost their dividing potential even if they were cultured in a medium containing no GJ-B-3. Although all the cancer cell lines tested responded significantly to GJ-B-3 treatment, PC3, MCF7, H292 are much more sensitive to GJ-B-3 treatment than others.

Tunnel experiments suggested that the possible mechanism underlying GJ-B-3-induced growth inhibition and killing of cancer cells, such as HepG2 cells, in vitro is to activate the HepG2 cell apoptosis pathway as demonstrated by caspase-8 fluorescent assay kit. After the HepG2 cells were treated with 10-20 μg/ml GJ-B-3, the caspase-8 activity increased gradually and reached the peak level at 18 hours compared with the control group, which also showed dose-dependent effect in that the increased concentration of GJ-B-3 also induced higher activity of the caspase-8. For example, the activity of caspase-8 reached 367.3±6.7 at the concentration (20 μg/ml) of GJ-B-3, while 286.6±6.3 at the lower concentration of 10 μg GJ-B-3/ml (P<0.01) compared with the even lower caspase-8 activity (76.5±5.3) in the non-treated controls. The apoptotic rate of HepG2 cells in GJ-B-3 treated groups was significantly higher than that in the non-treated control group (P<0.001). It is, therefore, suggested that GJ-B-3 can induce apoptosis of cancer cells in culture via caspase-8 signal transduction pathway following a concentration-dependent manner.

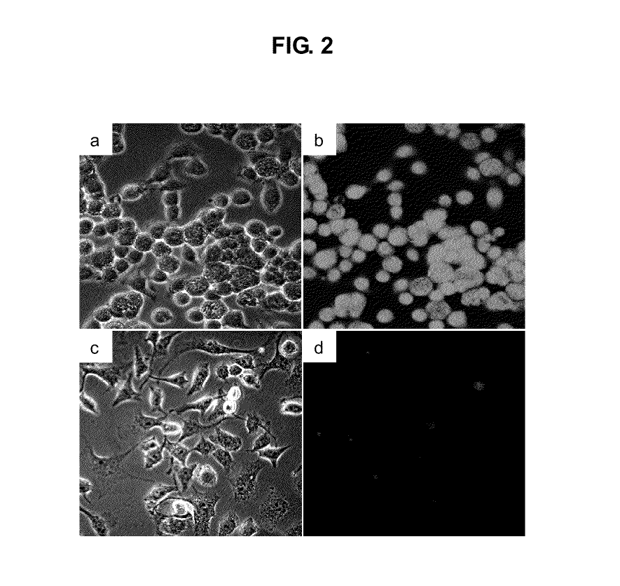

To further define the action underlying the anti-cancer effect of GJ-B-3, HepG2 cells were treated with GJ-B-3 at 20 μg/ml in a time-course manner to determine whether this bioactive fraction specifically induced HepG2 cell death via apoptosis or necrosis. HepG2 cells, which were treated with GJ-B-3 for 24 hours, showed active apoptosis and the fragmented DNA were labeled with fluorescence 12 dUTP in the nuclei. At the beginning of the treatment, the intensity of the green fluorescence was dim. However, more fluorescence was observed at 24 hours post treatment, indicating more cells were undergoing apoptosis (˜90%). In HepG2 cells treated with DMSO as negative control, no fluorescence was detected in the nuclei, due to the absence of fragmented DNA. As shown in FIG. 2, which demonstrates that GJ-B-3 induced apoptosis of cultured HepG2 cells. In panel a, GJ-B-3 treated HepG2 cells (24 h) is shown to have become smaller and more rounded under phase contrast microscope. Most of the treated cells in panel a showed strong green fluorescence (as shown in panel b), indicating their undergoing apoptosis. By comparison, in panel c, the cells were treated with 5% DMSO and their shapes were more irregular and no fluorescence was detected in the nuclei of the cells (as shown in panel d). The percentages of apoptotic cells after GJ-B-3 treatment increased in a time-dependent manner with >50% at 24 hours and 90% by 48 hours. By contrast, the non-treated control cells showed only approximately 5% of cell death via apoptosis.

### III. GJ-B-3 Induced Inhibition of Growth of Hepatic Tumor of HepG2 Origin

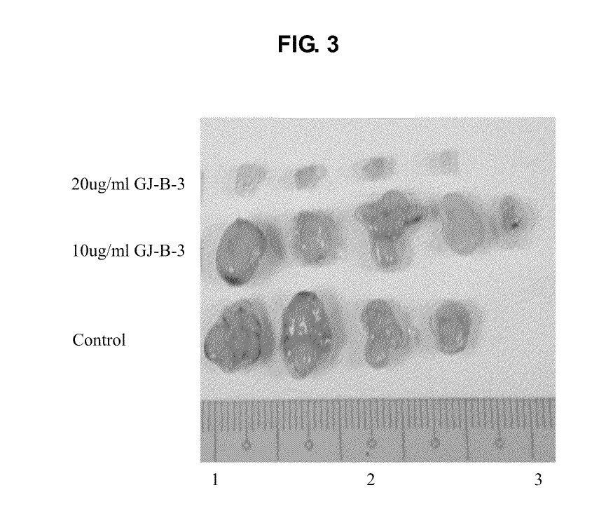

To evaluate the potential anti-tumor effect of GJ-B-3, tests were conducted with a hepatic cancer animal model. The result is shown in FIG. 3, which clearly demonstrated that the growth and spread of hepatic cancer cells, which had been inoculated beneath the skin in naked mice, were well controlled and the sizes of the tumors were significantly reduced by the treatment of GJ-B-3 for 3 weeks through oral administration (100 mg-400/kg body weight/per day). Thus, experimental data obtained in the present invention have demonstrated that GJ-B-3 has broad spectra of anti-tumor effects including primary tumors and tumor metastases in several major cancers, including cancers of the prostate, liver, breast and lung.

### IV. GJ-B-3 Induced Apoptosis of Cancer Cells In Vivo

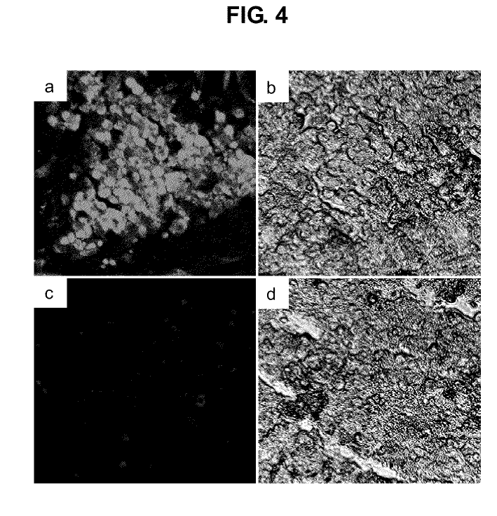

In order to demonstrate whether the GJ-B-3-induced cancer cell death in vivo was also via apoptosis as found in the cell culture system, HepG2 cells were inoculated beneath the skin of nude mice. GJ-B-3 was injected via the tail vein of the experimental animal one week after the inoculation of the cancer cells daily for two weeks. The result is shown in FIG. 4, which demonstrated that GJ-B-3 induced apoptosis of inoculated cancer cells in vivo. In panel b, the inoculated cancer cells from the treated animal were shown to have become shrinking, smaller and rounder. Significant more fluorescence was observed that was co-localized with the tumor cells in panel a, indicating that these tumor cells were significantly undergoing apoptosis. By comparison, the tumor cells from the animal treated with 5% DMSO showed significant larger size tumor cells (in panel d) and showed no fluorescence in the nuclei of tumor cells (panel c). Therefore, the in vivo experimental data demonstrated that in the treated animal, cancer became shrinking, smaller and rounder than prior to treatment. Significant more fluorescence was observed at the end of treatment that was co-localized with the tumor cells, thereby indicating that these tumor cells were undergoing apoptosis. This finding is consistent with the result obtained from in vitro studies. In comparison, the control tumor cells treated with 5% DMSO (the solvent of GJ-B-3) showed significant larger sized tumor cells and no fluorescence in the nuclei of tumor cells was observed.

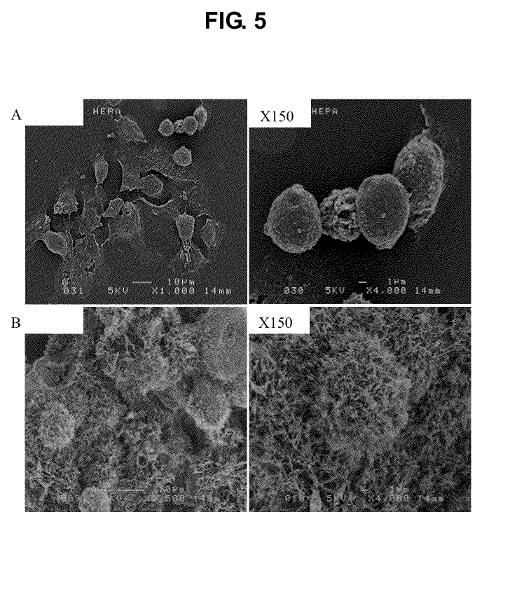

As shown in FIG. 5, under the electron microscope, the appearance of untreated HepG2 cells was irregular, the size of the tumor cells was significantly larger. There were rich mivrovilli on the surface of the untreated tumor cells. In comparison, the treated tumor cells became shrinking, smaller and rounder. There were almost no microvilli on the surface of the cells observed.

While there have been described and pointed out fundamental novel features of the invention as applied to a preferred embodiment thereof, it will be understood that various omissions and substitutions and changes, in the form and details of the embodiments illustrated, may be made by those skilled in the art without departing from the spirit of the invention. The invention is not limited by the embodiments described above which are presented as examples only but can be modified in various ways within the scope of protection defined by the appended patent claims.

### REFERENCES

1. Cragg G M, Newman D J: Medicinals for the millennia: The historical record Ann NY Acad Sci 953:3-25, 2001
2. Newman D J, Cragg G M, Snader K M: The influence of natural products on drug discovery. Nat Prod Rep 17:215-234, 2000
3. Cragg G M, Newman D J, Snader K M: Natural products in drug discovery and development. J Nat Prod 60:52-60, 1997
4. Perry, L. M. Medicinal Plants of East and Southeast Asia. Cambridge, Mass.: MIT Press, 133, (1980).

What is claimed is:

1. A pharmaceutical composition, comprising an extract of Geum japonicum and useful for for the treatment of cancers and tumors.

2. A method for the treatment of cancers and tumors without affecting healthy cells by using a composition of claim 1.

3. A method of selectively inducing apoptosis in cancer cells by contacting the cells with an extract from Geum japonicum
